(19) United States
(12) Patent Application Publication
(10) Pub. No.: US 2013/0109807 A1
(43) Pub. Date: May 2, 2013

(54) REMOVAL OF VIRUCIDAL AGENTS IN MIXED MODE CHROMATOGRAPHY

(71) Applicant: Bio-Rad Laboratories, Inc., Hercules, CA (US)

(72) Inventor: Peter S. GAGNON, Hercules, CA (US)

(73) Assignee: Bio-Rad Laboratories, Inc., Hercules, CA (US)

(21) Appl. No.: 13/654,802

(22) Filed: Oct. 18, 2012

Related U.S. Application Data
(60) Provisional application No. 61/551,735, filed on Oct. 26, 2011.

(51) Int. Cl. C07K 1/20; C08F 26/10

(52) U.S. Cl. 525/54.1; 530/416; 530/387.1

(57) ABSTRACT

The present invention provides chromatography methods for removing a virucidal agent from a target protein such as an antibody. The method uses a hydrophobic, negatively charged mixed mode support that has high affinity for both the target protein and the virucidal agent. The method provides conditions that favor dissociation of the virucidal agent from the protein, allowing the virucidal agent to bind strongly to the support. The target protein is then eluted from the support under conditions such that the virucidal agent remains bound to the support.

### CROSS-REFERENCE TO RELATED PATENT APPLICATIONS

The present patent application claims benefit of priority to U.S. Provisional Patent Application No. 61/551.735, filed Oct. 26, 2011, which is incorporated by reference for all purposes.

### BACKGROUND OF THE INVENTION

Natural and recombinant proteins produced by in vivo or in vitro methods require treatment with virucidal conditions or compounds to ensure the safety of patients receiving therapy based on those proteins. Many virucidal agents are toxic. A complication of virucidal treatment is that the virucidal agents themselves may form stable associations with treated protein products. These associations may make it difficult or impossible to completely remove the virucidal agent from the protein preparation.

### BRIEF SUMMARY OF THE INVENTION

Methods of removing a cationic or neutral virucidal agent from a biomolecule preparation are provided.

In some embodiments, the methods comprise, contacting a biomolecule preparation comprising a target biomolecule and the virucidal agent to a mixed mode support, wherein the mixed mode support has hydrophobic and negatively charged moieties, under conditions to allow the target biomolecule and the virucidal agent to bind to the support; and eluting the biomolecule target from the support under conditions such that the virucidal agent remains bound to the support. In other embodiments, detailed below, the virucidal agent is eluted before elution of the biomolecule.

In some embodiments, the biomolecule is a protein. In some embodiments, the protein is an antibody. In some embodiments, the antibody is an IgM or IgG antibody.

In some embodiments, the mixed mode support comprises p-aminohippuric acid attached to a pore matrix produced by polymerization of monomers 3-allyloxy-1,2-propanediol, vinylpyrrolidinone and crosslinked with N,N′-methylenebisacrylamide. In some embodiments, the mixed mode support comprises a mixed mode ligand comprising a 2-(benzoylamino) butanoic acid substituent. In some embodiments, the mixed mode support comprises a mixed mode ligand selected from the group consisting of hexanoic acid, phenylalanine, and a t-butyl ether derivative of polymethacrylate.

In some embodiments, the biomolecule preparation has a pH of between 4-6 when contacted to the mixed mode support. In some embodiments, the biomolecule preparation has a pH of between 4.5-5.5 when contacted to the mixed mode support.

In some embodiments, the method further comprises, between the contacting and the eluting, washing the support with a wash solution. In some embodiments, the wash solution comprises at least 0.1 M (e.g., at least 0.5 M, 1.0 M, 1.5 M, 2 M, etc.) sodium.

In some embodiments, the eluting comprises raising the pH of solution in contact with the target biomolecule bound to the support.

In some embodiments, the virucidal agent is selected the group consisting of polyethyleneimine, ethacridine, chlorohexidine, benzalkonium chloride, and methylene blue. In some embodiments, the virucidal agent is tri(n-butyl)phosphate (TNBP).

In some embodiments, the method further comprises eluting the virucidal agent after elution of the target biomolecule.

In some embodiments, the methods comprise contacting a biomolecule preparation comprising a target biomolecule and the virucidal agent to a mixed mode support, wherein the mixed mode support has hydrophobic and negatively charged moieties, under conditions to allow the target biomolecule and the virucidal agent to bind to the support; washing the support with a wash solution such that the virucidal agent is removed but the target molecule remains bound to the support; and eluting the biomolecule target from the support.

In some embodiments, the wash solution comprises at least 0.1 M (e.g., at least 0.5 M, 1.0 M, 1.5 M, 2 M, etc.) sodium.

In some embodiments, the biomolecule is a protein. In some embodiments, the protein is an antibody. In some embodiments, the antibody is an IgM or IgG antibody.

In some embodiments, the mixed mode support comprises p-aminohippuric acid attached to a pore matrix produced by polymerization of monomers 3-allyloxy-1,2-propanediol, vinylpyrrolidinone and crosslinked with N,N′-methylenebisacrylamide. In some embodiments, the mixed mode support comprises a mixed mode ligand comprising a 2-(benzoylamino) butanoic acid substituent. In some embodiments, the mixed mode support comprises a mixed mode ligand selected from the group consisting of hexanoic acid, phenylalanine, and a t-butyl ether derivative of polymethacrylate.

In some embodiments, the biomolecule preparation has a pH of between 4-6 when contacted to the mixed mode support. In some embodiments, the biomolecule preparation has a pH of between 4.5-5.5 when contacted to the mixed mode support.

In some embodiments, the eluting comprises raising the pH of solution in contact with the target biomolecule bound to the support.

In some embodiments, the virucidal agent is selected the group consisting of polyethyleneimine, ethacridine, chlorohexidine, benzalkonium chloride, and methylene blue. In some embodiments, the virucidal agent is tri(n-butyl)phosphate (TNBP).

Also provided are mixed mode supports in contact with a target biomolecule and a virucidal agent, wherein the mixed mode support has hydrophobic and negatively charged moieties.

In some embodiments, the biomolecule is a protein. In some embodiments, the protein is an antibody. In some embodiments, the antibody is an IgM or IgG antibody.

In some embodiments, the mixed mode support comprises comprises p-aminohippuric acid attached to a pore matrix produced by polymerization of monomers 3-allyloxy-1,2-propanediol, vinylpyrrolidinone and crosslinked with N,N′-methylenebisacrylamide. In some embodiments, the mixed mode support comprises a mixed mode ligand comprising a 2-(benzoylamino) butanoic acid substituent. In some embodiments, the mixed mode support comprises a mixed mode ligand selected from the group consisting of hexanoic acid, phenylalanine, and a t-butyl ether derivative of polymethacrylate.

In some embodiments, a solution comprising the virucidal agent and target biomolecule is in contact with the mixed mode support and the solution has a pH of between 4-6. In some embodiments, the solution has a pH of between 4.5-5.5 when contacted to the mixed mode support.

In some embodiments, the virucidal agent is selected the group consisting of polyethyleneimine, ethacridine, chlorohexidine, benzalkonium chloride, and methylene blue. In some embodiments, the virucidal agent is tri(n-butyl)phosphate (TNBP).

### DEFINITIONS

“Antibody” refers to an immunoglobulin, composite, or fragmentary form thereof. The term may include but is not limited to polyclonal or monoclonal antibodies of the classes IgA, IgD, IgE, IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” may also include composite forms including but not limited to fusion proteins containing an immunoglobulin moiety. “Antibody” may also include antibody fragments such as Fab, F(ab′)2, Fv, scFv, Fd, dAb, Fc and other compositions, whether or not they retain antigen-binding function.

“Mixed mode chromatography support” refers to a solid phase chromatographic support that employ multiple chemical mechanisms to adsorb proteins or other solutes. The solid phase can be a porous particle, nonporous particle, membrane, or monolith. Examples include but are not limited to chromatographic supports that exploit combinations of cation exchange (i.e., in which the support is anionic) and hydrophobic interaction.

“Target biomolecule” refers to a biomolecule, or molecule of biological origin, for purification according to the methods of the present invention. Target molecules include, but are not limited to, proteins. Examples of proteins include but are not limited to antibodies, enzymes, growth regulators, clotting factors, and phosphoproteins.

“Biomolecule preparation” and “biological sample” refer to any composition containing a target molecule of biological origin (a “biomolecule”) that is desired to be purified. In some embodiments, the target molecule to be purified is an antibody or non-antibody protein.

The term “detergent” refers to amphipathic, surface active, molecules with polar (water soluble) and nonpolar (hydrophobic) domains. Detergents bind strongly to hydrophobic molecules or molecular domains to confer water solubility. Examples of detergents are described in U.S. Pat. No. 5,883,256. In contrast to the use of detergents in U.S. Pat. No. 5,883,256, the present invention dissociates complexes of target molecules and virucidal agents by differential affinity to chromatography supports.

### DETAILED DESCRIPTION OF THE INVENTION

I. Introduction

It has been surprisingly discovered that an antibody can be purified from a positively charged or neutral virucidal agent, including virucidal agents that have formed a complex with the antibody, by contacting the antibody and the virucidal agent with a mixed mode support comprising hydrophobic and negatively charged moieties, and eluting the bound antibody from the support such that the antibody is substantially free of the virucidal agent. Under appropriate conditions, the positively charged or neutral virucidal agent will have significantly higher affinity for the mixed mode support than for the antibody, thereby disassociating the virucidal agent from complexes with the antibody. The chromatography is operated in a bind-elute format. Since the virucidal agent has higher affinity for the solid phase than the antibody, the virucidal agent abandons the antibody in favor of the solid support. In other words, the solid phase competitively dissociates the agent from the antibody.

When bind-elute conditions are employed, additional wash steps can be included to remove other contaminants or as a separate independent anti-viral step. For example, it has been discovered that in some embodiments the antibody bound to the support can be washed in high salt conditions. Such embodiments can include, for example loading the antibody under low pH conditions, thereby providing conditions of high affinity between the antibody and the support, and allowing tolerance of high salt conditions without antibody elution. In such circumstances, the antibody can be subsequently eluted (e.g., by raising the pH) following the wash, under conditions in which the virucidal agent remains bound to the support. While the initial discovery was made in the context of an antibody, it is believed that the methods can be adapted for the purification of other biomolecules.

In addition to the removal of virucidal agents from biomolecule preparations, it should also be noted that the methods of the invention can be adapted to function as two separate anti-viral steps. Removal and inactivation of virus is an increasing concern among health regulatory agencies worldwide. Some products such as IgG antibodies are fairly tolerant of virus inactivation at pH values below 3.8. However, many protein therapeutics (including but not limited to Factor VIII and IgM) are inactivated or aggregated by such conditions, and require the application of antiviral treatments under milder conditions. However, many treatments carried out under milder conditions are not effective to inactivate certain viral classes such as nonenveloped retrovirus. By adapting one or more wash steps in the bind-elute mode to involve a high salt concentration, the wash step itself can function to both remove contaminants and to act as an antiviral step due to the anti-viral effects of the high salt concentration.

II. Methods

The methods of the present invention use a mixed mode chromatography support comprising hydrophobic and negatively charged moieties to purify a target molecule from positively-charged or neutral virucidal agents, including such agents that have formed a complex with the target biomolecule. The methods can involve an initial incubation step in which the virucidal agent is incubated with the biomolecule preparation for a suitable time and under suitable conditions as known in the art to allow for the virucidal agent to bind, disrupt, or otherwise inactivate viruses present in the preparation. After the incubation, the preparation containing the virucial agent can be contacted to the mixed mode chromatography support as described herein, either directly or after one or more initial purification steps.

### Contacting Step

In some embodiments, prior to contacting the sample comprising the target molecule with the mixed mode support (e.g., mixed mode column), the chemical environment inside the column is equilibrated. In some embodiments, the mixed mode support can be equilibrated to establish an appropriate pH, conductivity, and/or concentration of salts. Equilibration of the support is accomplished, for example, by flowing an equilibration buffer containing appropriate reagents through the column. Buffering compounds may include but are not limited to MES, HEPES, BICINE, imidazole, and Tris.

In some embodiments, the mixed mode column is equilibrated to a pH of between 3 and 6. In some embodiments, the mixed mode column is equilibrated to a pH of between 4-6. In some embodiments, the mixed mode column is equilibrated to a pH of between 4.5-5.5. In some embodiments, the mixed mode column is equilibrated to a pH of about 5. Such lower-than-neutral pH values can improve binding of the target protein (e.g., antibody) to the negatively-charged chromatography support. Low pH minimizes the negative charge on the protein, which tends to weaken electrostatic interactions between the protein and the multivalent cationic virucidal agent, thereby favoring binding of the positively charged agent to the negatively charged support. Low pH simultaneously promotes strong protein binding, which translates into high dynamic binding capacity of the support. Therefore, in some embodiments, the lowest pH that the stability/bioactivity of the target biomolecule will tolerate is used. If the target molecule (e.g., an IgG) will tolerate the conditions, it can be desirable to use a pH close to, or preferably below, 3.75, because the pH itself mediates a strong antiviral effect.

The sample comprising the target molecule can also be equilibrated to conditions compatible with the column equilibration buffer before adding the sample to the column. In some embodiments, the preparation can be equilibrated by adjusting the pH, concentration of salts, and other compounds as desired. In some embodiments, the sample is equilibrated to a pH of between 4-6. In some embodiments, the sample is equilibrated to a pH of between 4.5-5.5. In some embodiments, the sample is equilibrated to a pH of 5. In some (rarer) embodiments, the equilibration conditions can include salt at a level that allows for target molecule binding of the support. For example, in some embodiments, IgGs will bind the mixed mode support at up to 4 M NaCl at pH values of 4.5 or lower. As each target will vary in its precise chromatographic attributes, it can be helpful to perform a 2-D study varying salt and pH to find the lowest pH and highest salt concentration where the protein binds with acceptable capacity and does not become inactivated.

After the column and biomolecule preparation have been equilibrated, the biomolecule preparation can be contacted to the mixed mode support (e.g., column) under conditions that allow for the target molecule (which may be complexed with a virucidal agent) to bind to the mixed mode support. Due to the affinity of the negatively charged/hydrophobic mixed mode support, the positively-charged or neutral virucidal agent will also bind strongly to the support. Indeed, this binding will be sufficiently strong to disrupt target biomolecule/virucidal agent complexes that might have formed. In some embodiments, the biomolecule preparation can be contacted with the column at a linear flow velocity in the range of, but not limited to, about 50-600 cm/hr, and in some embodiments, about 150-300 cm/hr. Appropriate flow velocity can be determined by the skilled artisan.

### Bind-Elute: Washing Step

Following binding of the target molecule to the mixed mode solid support, the bound target molecule is optionally washed with one or more agents under conditions in which the target molecule remains substantially bound to the solid support, where the presence and amount of the agent functions as an anti-viral agent condition, displaces complexed virucidal agent from the target molecule, and/or removes other contaminants (e.g., in the case where the target molecule is an antibody, then DNA, endotoxin, residual host-cell proteins, and leached protein A are some undesirable contaminants). For example, if complexes exist between a target IgG and DNA, high NaCl will weaken those complexes and the negative charge of the mixed mode may push DNA off and out of the support. This same effect can also occur to remove endotoxins and certain viruses embodying strong electronegativity.

A variety of washing agents can be used as an anti-viral wash, to displace or dissociate the virucidal agent from the target biomolecule, or both. A sufficient amount of these agents can be used in the wash to achieve an anti-viral effect, e.g., to inactivate at least 50%, 90%, 95%, 99%, 99.9%, or more of virus present.

In some embodiments, the agent is sodium chloride or another salt. At sufficient concentrations, sodium chloride can function as an antiviral agent. For example, in some embodiments, the sodium concentration is between 1-5 M, and in some embodiments, can be used up to saturation concentration. Washes at high salt concentrations suppress electrostatic interactions between target proteins and the virucidal agent, which further favors binding of the positively charged agent to the negatively charged support. At such salt concentrations, the electrostatic interaction between the agent and the negatively charged support no longer exists. In some embodiments, the virucidal agent is strongly hydrophobic, and the elevated salt concentration favors binding of the virucidal agent to the hydrophobic component/functionality of the mixed mode support, which further favors dissociation of the virucidal agent from the target protein. In addition, to the extent that a protein tends to bind strongly to cation exchangers, which includes most IgGs and many other proteins, and to the extent the pH is kept low enough to maintain binding, one can apply detergent washes, nonionic chaotropes (e.g., urea), arginine (e.g., 100-200 mM, 50-300 mM, 25-300 mM, 10-500 mM), and/or organic solvents (alcohols, glycols (e.g., ethylene glycol, propylene glycol), DMSO, DMF) to inactivate virus. Alcohols in particular are useful agents for disrupting non-enveloped retrovirus. Notably, high salt washes and alcohol-containing washes can be incompatible in some circumstances, and thus if both are desired they can be performed in series (one after the other). Indeed, the present methods are not limited to a single wash step but can include 2, 3, or more washes, including those described above.

In some embodiments, the pH of the wash solution is the same as the pH of the equilibration solution. In some embodiments, for example, the pH of the wash solution is between 4-6, between 4.5-5.5, or about 5. In cases where the protein is sufficiently tolerant of acidic conditions, an even lower pH is possible, though few proteins will tolerate a pH lower than 3. In some embodiments, the wash step comprises contacting the support with a solution of about pH 3.75 for at least 30 or 60 minutes, which is a current regulatory minimal low pH virucidal step. The low pH step can be combined with high NaCl or arginine to compound the virucidal effect, at least in circumstances win which the target molecule is not eluted. In situations in which the target molecule is not stable at pH 3.75, slightly higher pH values can be used to inactivate many viruses, though in some cases longer exposure to the conditions are needed to generate the same level of virus killing. The compound effect of the NaCl and/or arginine in these circumstances can improve the efficiency of anti-viral activity in these circumstances.

### Bind-Elute: Eluting Step

The target molecule can be eluted from the mixed mode support after the washing step described above. In some embodiments, the target molecule is eluted by raising the pH of the solution in contact with the target molecule bound to the support. For example, in some embodiments, the target biomolecule is eluted with a pH gradient, for example from equilibration pH up to pH 8.5 or higher. Alternatively, or in combination, in some embodiments, the target biomolecule can be eluted with salt gradients from nil to up to 2 M NaCl or higher. Other elution conditions can also be applied as desired, including, e.g., elution by inclusion of secondary modifiers, such as urea. It will be understood that every target biomolecule, and thus that elution conditions, can be determined by testing. Some exemplary elution gradients include, but are not limited to:

### pH Gradients

20 mM phosphate, 20 mM citrate, pH 4.5 to 20 mM phosphate, 20 mM citrate, pH 7.5.

Same in the presence of 100 mM NaCl

Same in the presence of 500 mM NaCl

### Salt Gradients

50 mM acetate, pH 4.5 (or for example starting as low as 3.5) to same plus 500 mM NaCl

Same to endpoint of 1 M NaCl

Same to endpoint of 2 M NaCl

Same to endpoint of 3 M NaCl

In some embodiments, the target molecule is eluted from the solid support while the virucidal agent (or other contaminants) from which the target molecule was dissociated are bound to the solid support. In some embodiments, the target molecule is eluted from the solid support after the contaminants from which the target molecule was dissociated are washed from the solid support. In some embodiments, the virucidal agent is washed from the solid support during the wash step(s). As an example of the latter case, in some embodiments, TNBP, benzalkonium chloride, or methylene blue will elute in a combination salt/detergent or salt/alcohol wash at low pH.

In some embodiments, the target biomolecule is an antibody or other protein and the preparation is equilibrated to pH 4-6 (e.g., about pH 5) before being contacted to the mixed mode column. In some embodiments comprising these conditions, the bound antibody/protein will not elute from the support, even at high salt concentrations. Thus, in some embodiments, elution can be triggered by raising the pH of the solution in contact with the chromatography support and bound antibody/protein. It will be appreciated that the elution conditions will depend on the electrostatic and hydrophobic properties of each individual target protein. For example, at a constant pH, the salt concentration at which a given protein molecule elutes from the mixed mode support will vary based on the properties of the protein. In some embodiments, the protein will remain bound to the support at 0.1 M, 0.25 M, 0.5 M, 0.75 M, 1.0 M, 1.5 M, or 2.0 M NaCl or above, or any concentration in between.

In some embodiments, at least 50%, 60%, 70%, 80%, 90%, 95%, or more of the target molecule bound to the solid support is eluted in the elution step.

In some embodiments, the target molecule that is eluted from the solid support is substantially free of virucidal agents and other contaminants.

Whether complexed contaminants such as a virucidal agent have been dissociated from the target molecule, and the extent to which complexed contaminants have been dissociated from the target molecule, can be determined. In some embodiments, it is determined by generating elution profiles for the chromatography run and looking at the pattern and/or size of peaks produced during the purification process. Additionally, when the target molecule or contaminant is DNA or protein, the removal of contaminants from the target molecule can be evaluated by measuring the A254 or A260 (absorbance at 260 nm; DNA) and/or A280 (absorbance at 280 nm; protein) profiles. In some embodiments, the removal of virucidal agents can be evaluated by measuring the A260, A280 and A365 (absorbance at 365 nM; e.g., ethacridine) profiles.

Optionally, the virucidal agent can be eluted from the mixed mode support after the target molecule is eluted. In some embodiments, the virucidal agent is eluted by increasing the salt concentration in a solution contacted with the mixed mode support. In some embodiments, the salt concentration is increased to a range of about 2-5 M NaCl (or equivalent concentration if other salts are used). In some embodiments, the virucidal agent is eluted by contacting the mixed mode support with an ionic chaotropic agent, such as guanidine. In some embodiments, the virucidal agent is eluted by contacting the mixed mode support with 3-6 M guanidine. In some embodiments, it is not economical to remove virus inactivating agents to an extent sufficient to restore the mixed mode to its original conditions. In such cases, and even where not strictly required, the mixed mode can be used on a disposable basis.

### Optional Additional Steps

The present invention may be combined with other purification methods to achieve higher levels of purification. Examples include, but are not limited to, other methods commonly used for purification of antibodies, such as protein A and other forms of affinity chromatography, anion exchange chromatography, apatite chromatography (e.g., hydroxyapatite and fluorapatite), cation exchange chromatography, hydrophobic interaction chromatography, immobilized metal affinity chromatography, and additional mixed mode chromatography methods. Other options, include, but are not limited to precipitation, crystallization, and/or liquid partitioning methods.

III. Target Biomolecules

The present invention provides methods of purifying a target biomolecule from a biological sample. In some embodiments, the target biomolecule in the biological sample is complexed with one or more virucidal agents.

Target biomolecules of the present invention include any biological molecule that may be purified using mixed mode chromatography. Examples of target biomolecules include, but are not limited to, proteins (e.g., antibodies, non-antibody therapeutic proteins, enzymes, growth regulators, clotting factors, and phosphoproteins).

In some embodiments, the target molecule is an antibody or antibody fragment. In some embodiments, the antibody is an IgG, IgM, IgA, IgD, or IgE. In some embodiments, the target biomolecule is a Fc-fusion protein. Antibody preparations for use in the present invention can include unpurified or partially purified antibodies from natural, synthetic, or recombinant sources. Unpurified antibody preparations may come from various sources including, but not limited to, plasma, serum, ascites fluid, milk, plant extracts, bacterial lysates, yeast lysates, or conditioned cell culture media. Partially purified preparations may come from unpurified preparations that have been processed by at least one chromatography, precipitation, other fractionation step, or any combination of the foregoing.

Any antibody preparation can be used in the present invention, including unpurified or partially purified antibodies from natural, synthetic, or recombinant sources. Unpurified antibody preparations can come from various sources including, but not limited to, plasma, serum, ascites, milk, plant extracts, bacterial lysates, yeast lysates, or conditioned cell culture media. Partially purified preparations can come from unpurified preparations that have been processed by at least one chromatography, precipitation, other fractionation step, or any combination of the foregoing. In some embodiments, the antibodies have not been purified by protein A affinity prior to purification.

The invention can be of particular interest in purification of proteins that are sensitive to low pH, which is one industry-standard method of virus reduction. Many recombinant proteins (including but not limited to clotting factors, including Factor VIII and von Willebrand Factor, and IgM antibodies) are highly labile and do not survive low pH treatment and thus are good candidates for the methods of the invention, and in particular those in which the wash step includes a second anti-viral agent. Moreover, some target proteins or other biomolecules too large to support reduction of non-enveloped viruses by filtration methods because the hydrodynamic radius of the virus is the same as the target biomolecule. These biomolecules are thus also particularly good candidates for use in the present methods.

IV. Virucidal Agents

The present invention provides methods of removing a virucidal agent from a biological sample. In some embodiments, the methods are useful for dissociating one or more virucidal agents that are associated with a target molecule in order to enhance the purification of the target molecule. In some embodiments, the virucidal agent is positively charged. In some embodiments, the virucidal agent is neutral (not-charged). In some embodiments, the virucidal agent is polyethyleneimine (PEI), ethacridine, chlorhexidine, benzalkonium chloride, methylene blue, or tri(n-butyl)phosphate (TNBP). In some embodiments, the virucidal agent is a multivalent cationic agent such as those described in U.S. Pat. No. 6,831,066 to Bernhardt.

V. Kits

In another embodiment, the invention provides a kit for use in the methods described herein. A kit can optionally include written instructions or electronic instructions (e.g., on a CD-ROM or DVD) as well as packaging material. In some embodiments, the kits comprise a prepacked mixed mode chromatography support comprising hydrophobic and negatively charged moieties and a virucidal agent. Other reagents described herein in the context of the methods can also optionally be included in the kits. For instance, the kits can comprise one or more premixed buffer concentrates.

VI. Mixed-mode chromatography

In some embodiments, the mixed-mode chromatography support exploits a combination of cation exchange (i.e., negatively-charged moieties on the mixed mode support) and hydrophobic interaction functionalities, optionally with potential for hydrogen bonding and pi-pi bonding. A variety of support matrices can be used according to the present invention. Exemplary supports include those that comprise weakly hydrophobic functional groups. For example, the mixed mode ligand comprises a hydrophobic ligand but is not sufficiently hydrophobic to denature proteins. For example, in some embodiments, the support will comprise aliphatic moieties with 6 carbons or fewer, and/or aromatic moieties with a single 6-carbon ring. In some embodiments, the ligand is as displayed in Formula I (known commercially as “Capto MMC™” (available from GE Healthcare)), which comprises a 2-(benzoylamino) butanoic acid substituent.

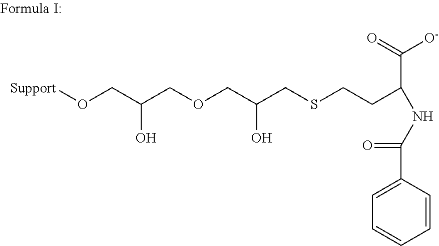

The Capto MMC ligand is described in, for example, Manufacturer data sheet GE Health Care (11-0035-45AA) Capto Adhere, Manufacturer data sheet GE Health Care (28-9078-88AA) Capto MMC and patent application EP 07114856.3. Other commercial examples of such supports include, but are not limited to, Macroprep S, Macroprep CM, and Macroprep t-butyl (available from Bio-Rad, Inc.). Additional exemplary polymers and functional groups for mixed mode supports are described in U.S. Pat. No. 6,423,666.

Structural groups that are useful as hydrophobic functionalities in the ligands described herein include aromatic and substituted aromatic groups. Phenyl and biphenyl groups, particularly phenyl groups, are common examples of aromatic groups and are used in certain embodiments herein. Suitable substituents are those that retain the hydrophobic character of the aromatic group; examples include certain alkyl groups such as hexyl. Substituents that creater steric hindrance to the immunoglobulins are less preferred. Structural groups that are useful as weak cationic exchange functionalities include carboxylic acids and carboxylates, and cationic groups in general with plc, values in the range of 3.7-5.5. The hydrophobic and weak cationic exchange moieties can be joined by a chain, preferably a chain that contains no more than five atoms, excluding hydrogen atoms and substituents. Examples of such chains are peptide—containing chains, such as —R¹—C(O)—NH—R²— where R¹ and R² are alkyl groups and one or both of R¹ and R² can be absent. A specific example is —C(O)—NH—CH₂—. A ligand containing the latter linkage between a carboxylic functionality as the weak cation exchange group and a phenyl functionality as the hydrophobic group is benzoylamino acetic acid. In some embodiments, the weak cationic exchange and hydrophobic functionalities are incorporated in a ligand that is bound to a solid matrix that has pores whose median diameter is 0.5 micron or greater, with substantially no pores of less than 0.1 micron in diameter, and the ligand is coupled to the support matrix at the hydrophobic group on the ligand through a linkage of a chain of one to three atoms. In some embodiments, the ligand is a copolymer of 3-allyloxy-1,2-propanediol and vinyl pyrrolidinone crosslinked with N,N′-methylenebisacrylamide. In some embodiments, the ligand p-aminohippuric acid attached to a large pore matrix produced by polymerization of monomers 3-allyloxy-1,2-propanediol, vinylpyrrolidinone and crosslinked with N,N′-methylenebisacrylamide.

In some embodiments, the support matrix will be a hydrophilic polymer that allows for linkage of the ligand, optionally via a spacer. In some embodiments, the hydrophilic polymer is derivatized to contain functional groups suitable for any type of chromatographic separation.

In some embodiments, the base matrix of the support is hydrophilic and in the form of a polymer, e.g. a polymer that is insoluble and more or less swellable in water. Suitable polymers are polyhydroxy polymers, e.g. based on polysaccharides, such as agarose, dextran, cellulose, starch, pullulan, etc. and completely synthetic polymers, such as polyacrylic amide, polymethacrylic amide, poly(hydroxyalkylvinyl ethers), poly(hydroxyalkylacrylates) and polymethacrylates (e.g. polyglycidylmethacrylate), polyvinyl alcohols and polymers based on styrenes and divinylbenzenes, and copolymers in which two or more of the monomers corresponding to the above-mentioned polymers are included. Polymers, which are soluble in water, may be derivatized to become insoluble, e.g. by cross-linking and by coupling to an insoluble body via adsorption or covalent binding. Hydrophilic groups can be introduced on hydrophobic polymers (e.g. on copolymers of monovinyl and divinylbenzenes) by polymerisation of monomers exhibiting groups which can be converted to OH, or by hydrophilization of the final polymer, e.g. by adsorption of suitable compounds, such as hydrophilic polymers.

Other non-commercial mixed mode media include, for example, mixed-mode chromatography support exploiting a combination of cation exchange with hydrophobic interaction functionalities in the same ligand, or in a combination of ligands. Some examples of such ligands are described in, e.g., U.S. Pat. Nos. 7,008,542; 6,498,236; and 5,945,520.

In some embodiments, mixed-mode chromatography supports exploiting a combination of cation exchange with hydrophobic interaction functionalities can be used. For example, ligands comprising at least one acidic moiety such as a carboxyl group and also comprising at least one hydrophobic moiety such as a phenyl ring or an aliphatic hydrocarbon chain can be used.

In some embodiments, phenylalanine is covalently linked to a solid support. For example, the phenylalanine can be covalently linked to the solid support via the amine of phenylalanine. Phenylalanine can be linked to a solid support, for example, by nucleophilic replacement of a leaving group on the solid support, or by other chemistries known to those skilled in the art.

The secondary amino on the phenylalanine can be “capped” with an additional moiety to form a tertiary amine, thereby preventing or reducing the formation of cationic ammonium (and therefore formation of a zwitterion) at the pH at which the chromatography is performed.

In some embodiments, the amine is capped with an acetyl group. Those of skill in the art will appreciate there are a number of ways to acetylate the amine. In some embodiments, the beads are dried and then exposed to acetyl chloride. In some embodiments the beads are submitted to solvent exchange with acetone, rather than drying, and then exposed to acetyl chloride.

In another embodiment, a t-butyl ether derivative of a polymethacrylate is used as the mixed mode ligand. An example of this type of derivative is Macro-Prep t-butyl HIC™, which is commercially available from Bio-Rad, Inc. (Hercules, Calif.). T-butyl ether derivatives can be formed from polymeric beads comprising (1) glycidyl methacrylate groups. For example, a fraction of the ester groups on the polymer backbone can be hydrolyzed to carboxylic acid groups while the reaction of t-butoxide proceeds with the epoxide. This is illustrated in the following diagram.

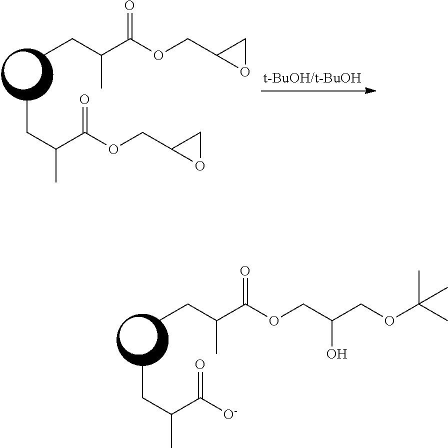

Macro-Prep t-Butyl HIC typically contains from 131-266 micromoles carboxyl groups/ml and 25-45 micromoles t-butyl groups/ml resin, though those of skill in the art will appreciate that the total amount and ratio of these two groups can be varied as desired.

In yet other embodiments, an alkyl acid (e.g., a carboxylic acid) is used as a mixed mode ligand. In some embodiments, the alkyl acid is an n-alkyl acid having between 4-10, 4-6, 4-8, 6-8, or 5-7 carbons, e.g., 2, 3, 4, 5, 6, 7, 8 carbons. As discussed in more detail in the examples, hexanoic acid can be used. Alternatively, in some embodiments, acetic acid, butanoic acid, octanoic acid, or decanoic acid are used.

Alkyl acids can be linked to a hydroxy-functionalized solid matrix. Halogenated alkyl acids can react directly with the hydroxy-functionalized solid matrix. For example, a bromoalkyl acid, such as 6-bromohexanoic acid, can be coupled to UNOsphere Diol. Reactions can be performed, e.g., with 1 M NaOH in the presence of excess bromoacid.

In yet another embodiment, Tosoh HIC media, which contains backbone carboxylate, similar to Macro-Prep t-Butyl HIC resin, is treated with NaOH, to form backbone carboxylates, producing a mixed mode similar to Macro-Prep t-Butyl HIC.

In some embodiments, the method is a preparative application for the purpose of obtaining a purified biological product (e.g., antibody or other protein) for research, diagnostic, therapeutic, or other applications. Such applications may be practiced at any scale, ranging from milligrams to kilograms of biological product per batch.

### EXAMPLES

### Example 1

### Removal of a Virucidal Agent from an Antibody Preparation

This example describes the use of a mixed mode support to remove a virucidal agent from an antibody preparation.

A biomolecule preparation comprising an IgM antibody was treated with the virucidal agent PEI-1300 (average molecular weight 1300 Daltons) at a concentration of 0.01%. A column comprising the ligand p-aminohippuric acid attached to a large pore matrix produced by polymerization of monomers 3-allyloxy-1,2-propanediol, vinylpyrrolidinone and crosslinked with N,N′-methylenebisacrylamide was equilibrated with 20 mM MES, 20 mM acetate, to pH 5.0. Sample pH was reduced to pH 4.75 by addition of 1 M acetate, pH 4.5, 5% vol:vol. then the sample was applied to the mixed mode support column. An intermediate 1 M NaCl wash was applied. The IgM antibody was eluted by raising the operating pH to 7.5 in a linear gradient ending at 20 mM Hepes. Antibody purity was estimated by analytical size exclusion chromatography (SEC) to be greater than 90%. Following elution of the IgM antibody, the virucidal agent was eluted by washing the column with 2 M NaCl. In other experiments it was removed with 3 M guanidine, pH 7.0. Elution of the virucidal agent was tracked by its high UV absorbance at 254 nm.

Another IgM preparation was treated with the virucidal agent ethacridine at a concentration of 0.02%. A column comprising the ligand p-aminohippuric acid attached to a large pore matrix produced by polymerization of monomers 3-allyloxy-1,2-propanediol, vinylpyrrolidinone and crosslinked with N,N′-methylenebisacrylamide was equilibrated with 50 M citrate, 50 mM phosphate, 500 mM NaCl, pH 4.5. The sample pH was reduced to 4.75 by addition of 1 M acetate, pH 4.5, 5% vol;vol and applied to the column. The ethacridine formed an intense narrow yellow band at the top of the column, The column was washed with 1 M NaCl at pH 4.5 to dissociated the ethacridine from the antibody and enhance virus inactivation, then eluted with a linear gradient from the equilibration buffer to 500 mM MNaCl, 50 mM citrate, 50 mM phosphate, pH 7.5. Ethacridine was subsequently eluted with 3 M guanidine. Antibody purity was estimated by anlalytical size exclusion chromatography (SEC) at greater than 90%.

In other experiments, the biomolecule preparation comprising an IgM antibody was treated with the virucidal agents ethacridine or chlorhexidine. The equilibration, wash, and antibody elution steps were as described above. When treating with ethacridine or chlorhexidine, the virucidal agent was eluted with 4 M guanidine instead of NaCl.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of removing a cationic or neutral virucidal agent from a biomolecule preparation, the method comprising,
contacting a biomolecule preparation comprising a target biomolecule and the virucidal agent to a mixed mode support, wherein the mixed mode support has hydrophobic and negatively charged moieties, under conditions to allow the target biomolecule and the virucidal agent to bind to the support; and
eluting the biomolecule target from the support under conditions such that the virucidal agent remains bound to the support.

2. The method of claim 1, wherein the biomolecule is a protein.

3. The method of claim 2, wherein the protein is an antibody.

4. The method of claim 3, wherein the antibody is an IgM or IgG antibody.

5. The method of claim 1, wherein the mixed mode support comprises hexanoic acid, phenylalanine, a t-butyl ether derivative of polymethacrylate, 2-(benzoylamino) butanoic acid, or p-aminohippuric acid attached to a pore matrix produced by polymerization of monomers 3-allyloxy-1,2-propanediol, vinylpyrrolidinone and crosslinked with N,N′-methylenebisacrylamide.

6. The method of claim 1, wherein the biomolecule preparation has a pH of between 4-6 when contacted to the mixed mode support.

7. The method of claim 1, further comprising, between the contacting and the eluting, washing the support with a wash solution.

8. The method of claim 7, wherein the wash solution comprises at least 0.1 M sodium.

9. The method of claim 1, wherein the eluting comprises raising the pH of solution in contact with the target biomolecule bound to the support.

10. The method of claim 1, wherein the virucidal agent is selected the group consisting of tri(n-butyl)phosphate (TNBP), polyethyleneimine, ethacridine, chlorohexidine, benzalkonium chloride, and methylene blue.

11. A method of removing a cationic or neutral virucidal agent from a biomolecule preparation, the method comprising,
contacting a biomolecule preparation comprising a target biomolecule and the virucidal agent to a mixed mode support, wherein the mixed mode support has hydrophobic and negatively charged moieties, under conditions to allow the target biomolecule and the virucidal agent to bind to the support;
washing the support with a wash solution such that the virucidal agent is removed but the target molecule remains bound to the support; and
eluting the biomolecule target from the support.

12. The method of claim 11, wherein the wash solution comprises at least 0.1 M sodium.

13. The method of claim 11, wherein the biomolecule is a protein.

14. The method of claim 13, wherein the protein is an antibody.

15. The method of claim 14, wherein the antibody is an IgM or IgG antibody.

16. The method of claim 11, wherein the mixed mode support comprises hexanoic acid, phenylalanine, a t-butyl ether derivative of polymethacrylate, 2-(benzoylamino) butanoic acid, p-aminohippuric acid attached to a pore matrix produced by polymerization of monomers 3-allyloxy-1,2-propanediol, vinylpyrrolidinone and crosslinked with N,N′-methylenebisacrylamide.

17. The method of claim 11, wherein the biomolecule preparation has a pH of between 4-6 when contacted to the mixed mode support.

18. The method of claim 11, wherein the eluting comprises raising the pH of solution in contact with the target biomolecule bound to the support.

19. The method of claim 11, wherein the virucidal agent is selected the group consisting of tri(n-butyl)phosphate (TNBP), polyethyleneimine, ethacridine, chlorohexidine, benzalkonium chloride, and methylene blue.

20. A mixed mode support in contact with a target biomolecule and a virucidal agent, wherein the mixed mode support has hydrophobic and negatively charged moieties.
